(19) United States
(12) Patent Application Publication
(10) Pub. No.: US 2025/0378625 A1
(43) Pub. Date: Dec. 11, 2025

(54) METHOD AND SYSTEM FOR OVERLAYPRESENTATION OF SKELETAL IMAGEBASED ON AUGMENTED REALITY

(71) Applicant: The Fourth Medical Center of the Chinese People's Liberation Army General Hospital, Beijing (CN)

(72) Inventors: Xiang CUI, Beijing (CN); Chi MA, Beijing (CN); Houchen LV, Beijing (CN); Yong XIE, Beijing (CN); Licheng ZHANG, Beijing (CN); Peifu TANG, Beijing (CN)

(73) Assignee: The Fourth Medical Center of the Chinese People's Liberation Army General Hospital, Beijing (CN)

(21) Appl. No.: 19/312,912

(22) Filed: Aug. 28, 2025

(30) Foreign Application Priority Data
Aug. 28, 2024 (CN) 202411193684.1

(51) Int. Cl. G06T 15/06; G06T 5/20; G06T 5/40; G06T 5/94; G06T 7/00; G06T 7/33; G06T 15/08; G06V 10/60; G06V 10/77; G06V 10/82

(52) U.S. Cl. CPC G06T 15/06; G06T 5/20; G06T 5/40; G06T 5/94; G06T 7/0012; G06T 7/344; G06T 15/08; G06V 10/60; G06V 10/7715; G06V 10/82; G06T 2207/10081; G06T 2207/10116; G06T 2207/20012; G06T 2207/20081; G06T 2207/20084; G06T 2207/30008; G06T 2207/30204

(57) ABSTRACT

The present disclosure provides a method and system for overlay presentation of a skeletal image based on augmented reality, relating to the technical field of smart medical systems. The method includes obtaining fracture imaging data for a fracture region of a patient, the fracture imaging data including a CT image, an X-ray image, and a light-field image; parsing a ray model corresponding to the light-field image, the ray model providing spatial propagation information of a light in the fracture region of the patient; determining a multimodal fusion feature corresponding to the fracture imaging data based on the ray model and a feature fusion network; reconstructing a three-dimensional model of a fracture part based on the multimodal fusion feature; and aligning and calibrating the reconstructed three-dimensional model of the fracture part with the fracture region of the patient in an actual surgical scene.

### CROSS-REFERENCE TO RELATED APPLICATIONS

This application claims priority to Chinese Patent Application No. 202411193684.1 with a filing date of Aug. 28, 2024. The content of the aforementioned application, including any intervening amendments thereto, is incorporated herein by reference.

### FIELD

The present disclosure relates to the field of smart healthcare systems, and more particularly to a method and a system for overlay presentation of a skeletal image based on augmented reality (AR).

### BACKGROUND

In fracture diagnosis and treatment, medical imaging technologies play a crucial role. Common imaging techniques include X-ray, computed tomography (CT), and magnetic resonance imaging (MRI). Although these technologies have been widely applied in clinical practice, they still exhibit significant limitations in the presentation of fracture images.

Traditional two-dimensional imaging techniques, such as X-ray images, can provide planar views of the fracture site, but lack depth perception. As a result, doctors should rely on their spatial imagination to interpret the three-dimensional (3D) structure of the fracture during diagnosis and treatment. This approach not only increases the risk of misdiagnosis but may also lead to inaccurate treatment plans, prolonging patient's recovery time. Although CT and MRI can provide more detailed cross-sectional images, they are still two-dimensional. Doctors need to piece together multiple cross-sectional images to reconstruct the 3D structure, which is time-consuming and prone to errors.

Moreover, during fracture surgeries, doctors typically devise surgical plans depending on preoperative imaging data, and then follow the image-guided plan during the procedure. However, current imaging technologies cannot provide real-time visual guidance. Doctors need to rely on their experience and memory of the images to judge the details during the surgery, which increases the complexity and risk of surgery, and may prolong the surgical time, adding to the patient's pain and medical costs.

At present, the industry has not yet provided a better technical solution to address these issues.

### SUMMARY

Embodiments of the present disclosure provide a method and a system for overlay presentation of a skeletal image based on augmented reality, which at least solves the problem in the current related art where two-dimensional imaging fails to provide real-time visual guidance for doctors.

In a first aspect, embodiments of the present disclosure provide a method for overlay presentation of a skeletal image based on AR, which is applied to an AR glass. The method includes: obtaining fracture imaging data for a fracture region of a patient, the fracture imaging data comprising a CT image, an X-ray image, and a light-field image; parsing a ray model corresponding to the light-field image, the ray model providing spatial propagation information of a light in the fracture region of the patient; determining a multimodal fusion feature corresponding to the fracture imaging data based on the ray model and a feature fusion network, the feature fusion network adopting a convolutional neural network. During a feature fusion process, the ray model is utilized as guidance information, and a ray consistency constraint is introduced into the feature fusion network to enable a consistency of a fused feature along a ray propagation path. The loss function L of the feature fusion network is defined as:

$$L = {λ}_{ray} {L}_{ray} + {λ}_{feat} {L}_{feat}$$

$${L}_{ray} = {\sum }_{p ∈ P} {‖ {F}_{fusion} ( p ) - R ( p ) ‖}^{2}$$

$${L}_{feat} = {\sum }_{f ∈ F} {‖ {F}_{fusion} ( f ) - {F}_{input} ( f ) ‖}^{2}$$

where L_{feat} represents a feature matching loss configured to enable a consistency between the fused feature and an input feature in a feature space; L_{ray} represents a ray consistency constraint loss configured to enable the consistency of the fused feature along the ray propagation path; λ_{ray} is a weight coefficient of the L_{ray}, λ_{feat} is a weight coefficient of the L_{feat}; p represents a point on the ray, P represents a set of all ray points, F_{fusion}(p) represents a fused feature at point p, R(p) represents a feature value of the ray model at the point p; f represents a point on a feature map, F represents a set of all points on the feature map, F_{fusion}(f) represents a fused feature at the point f, and Fᵢₙₚᵤₜ(f) represents an input feature at the point f;

the weights of respective convolution kernels in the feature fusion network are adjusted based on the ray model to enhance a ray consistency of features extracted by convolution:

$${F}_{c o n v} = σ ( {\sum }_{k = 1}^{K} {W}_{c o n v}^{( k )} * {F}_{input}^{( k )} + {b}^{( k )} )$$

$${W}_{c o n v}^{( k )} = {W}_{b a s e}^{( k )} + {α R}_{c o n v}^{( k )}$$

$${R}_{c o n v}^{( k )} = {\sum }_{i = 1}^{N} {w}_{i} {R}_{i}^{( k )}$$

$${w}_{i} = \frac{1}{Z} exp ( - \frac{1}{{θ}^{2}} {\sum }_{p ∈ {P}_{i}} {‖ {F}_{f u s i o n} ( p ) - {R}_{i} ( p ) ‖}^{2} )$$

where F_{conv} represents a feature map after convolution,

$${W}_{c o n v}^{( k )}$$

b^{(k)} respectively represent a weight and a bias of k-th convolution kernel,

$${F}_{input}^{( k )}$$

represents k-th channel of an input feature map, * represents a convolution operation, and σ represents a ReLU activation function;

$${W}_{b a s e}^{( k )}$$

represents a base weight of the k-th convolution kernel; α represents an adjustment coefficient configured to control an extent of influence of the ray model on a convolution kernel weight;

$${R}_{c o n v}^{( k )}$$

represents an adjusted weight guided by the ray model for the k-th convolution kernel; wWᵢ represents a weight of i-th ray, Pᵢ represents a set of all points on the i-th ray,

$${R}_{i}^{( k )}$$

represents a feature value of the i-th ray in the k-th convolution kernel, and N represents a number of rays; Rᵢ(p) represents a feature value of the i-th ray at a point p, θ represents a hyperparameter for controlling a weight distribution, and Z represents a normalization factor.

The method further includes reconstructing a three-dimensional model of a fracture part based on the multimodal fusion feature; and aligning and calibrating the reconstructed three-dimensional model of the fracture part with the fracture region of the patient in an actual surgical scene.

In a second aspect, embodiments of the present disclosure provide a system for overlay presentation of a skeletal image based on AR. The system includes: a data obtaining unit configured to obtain fracture imaging data for a fracture region of a patient, the fracture imaging data including a CT image, an X-ray image, and a light-field image; a ray model parsing unit configured to parse a ray model corresponding to the light-field image, the ray model providing spatial propagation information of a light in the fracture region of the patient; a fusion feature determination unit configured to determine a multimodal fusion feature corresponding to the fracture imaging data based on the ray model and a feature. fusion network, the feature fusion network adopting a convolutional neural network. During a feature fusion process, the ray model is utilized as guidance information, and a ray consistency constraint is introduced into the feature fusion network to enable a consistency of a fused feature along a ray propagation path. The loss function L of the feature fusion network is defined as:

$$L = {λ}_{ray} {L}_{ray} + {λ}_{feat} {L}_{feat}$$

$${L}_{r a y} = {\sum }_{p ∈ P} {‖ {F}_{f u s i o n} ( p ) - R ( p ) ‖}^{2}$$

$${L}_{feat} = {\sum }_{f ∈ F} {‖ {F}_{fusion} ( f ) - {F}_{input} ( f ) ‖}^{2}$$

where L_{feat} represents a feature matching loss configured to enable a consistency between the fused feature and an input feature in a feature space; L_{ray} represents a ray consistency constraint loss configured to enable the consistency of the fused feature along the ray propagation path; λ_{ray} is a weight coefficient of the L_{ray}, λ_{feat} is a weight coefficient of the L_{feat}; p represents a point on the ray, P represents a set of all ray points, F_{fusion}(p) represents a fused feature at point p, R(p) represents a feature value of the ray model at the point p; f represents a point on a feature map, F represents a set of all points on the feature map, F_{fusion}(f) represents a fused feature at the point f, and Fᵢₙₚᵤₜ(f) represents an input feature at the point f.

The weights of respective convolution kernels in the feature fusion network are adjusted based on the ray model to enhance a ray consistency of features extracted by convolution:

$${F}_{c o n v} = σ ( {\sum }_{k = 1}^{K} {W}_{c o n v}^{( k )} * {F}_{input}^{( k )} + {b}^{( k )} )$$

$${W}_{c o n v}^{( k )} = {W}_{b a s e}^{( k )} + α {R}_{c o n v}^{( k )}$$

$${R}_{c o n v}^{( k )} = {\sum }_{i = 1}^{N} {w}_{i} {R}_{i}^{( k )}$$

$${w}_{i} = \frac{1}{Z} exp ( - \frac{1}{{θ}^{2}} {\sum }_{p ∈ {P}_{i}} ‖ {F}_{fusion} ( p ) - {R}_{i} ( p ) {‖}^{2} )$$

where F_{conv} represents a feature map after convolution,

$${W}_{c o n v}^{( k )}$$

and b^{(k)} respectively represent a weight and a bias of k-th convolution kernel,

$${F}_{i n p u t}^{( k )}$$

represents k-th channel of an input feature map, * represents a convolution operation, and σ represents a ReLU activation function;

$${W}_{b a s e}^{( k )}$$

represents a base weight of the k-th convolution kernel; α represents an adjustment coefficient configured to control an extent of influence of the ray model on a convolution kernel weight;

$${R}_{c o n v}^{( k )}$$

represents an adjusted weight guided by the ray model for the k-th convolution kernel; wᵢ represents a weight of i-th ray, Pᵢ represents a set of all points on the i-th ray,

$${R}_{i}^{( k )}$$

represents a feature value of the i-th ray in the k-th convolution kernel, and N represents a number of rays; Rᵢ(p) represents a feature value of the i-th ray at a point p, θ represents a hyperparameter for controlling a weight distribution, and Z represents a normalization factor.

The system further includes a three-dimensional model construction unit configured to reconstruct a three-dimensional model of a fracture part based on the multimodal fusion feature; and an alignment and calibration unit configured to align and calibrate the reconstructed three-dimensional model of the fracture part with the fracture region of the patient in an actual surgical scene.

In a third aspect, embodiments of the present disclosure provide an electronic device, which includes: at least one processor, and a memory communicatively connected to the at least one processor, where the memory stores instructions executable by the at least one processor, and the instructions, when executed, cause the at least one processor to perform the steps of the aforementioned method.

In a fourth aspect, embodiments of the present disclosure provide a storage medium, where one or more program codes including executable instructions are stored. The program code can be read and executed by an electronic device (including but not limited to a computer, a processor, a server, or a network device, etc.) to perform the steps of the method described above in the present disclosure.

In a fifth aspect, embodiments of the present disclosure further provide a computer program product, which includes a computer program stored on a storage medium. The computer program includes program instructions that, when executed by a computer, enable the computer to perform the steps of the aforementioned method.

By means of the method, the system, the electronic device, and the non-transitorycomputer-readable storage medium for overlay presentation of a skeletal image based on augmented reality provided in the present disclosure, comprehensive visualization and real-time guidance for the fracture part can be achieved through multimodal data fusion, innovative 3D reconstruction techniques, and real-time AR presentation. At least the following technical effects can be provided.

(1) By fusing the CT image, X-ray image, and light-field image, and multimodal feature extraction is performed by using a ray model and a feature fusion network, with the introduction of ray consistency constraint to enable the consistency of the fused feature along the ray propagation path. This not only enhances the accuracy of the fused feature but also guarantees the consistency of different imaging data during the reconstruction process. As a result, the precision of 3D reconstruction of the fractured part can be significantly improved, allowing doctors to intuitively observe the 3D details of the fracture part, and overcoming the limitation of traditional 2D imaging that lacks depth perception.

(2) The advantages of various imaging data can be integrated by utilizing the multimodal fusion feature, addressing the limitations of any single modality. CT image can provide high-resolution cross-sectional information, X-ray image can provide rapid planar views, and light-field image can provide ray propagation data. By fusing the information, a more comprehensive and accurate 3D structure of the fracture part can be obtained.

(3) By aligning and calibrating the reconstructed 3D fracture model with the patient's fracture region in the actual surgical scene using AR glasses, the doctor can view the 3D image of the fracture region in real time during the operation, thereby achieving real-time visual guidance. This greatly reduces the need for doctors to rely on their own personal experience and image memory, simplifies the complexity and risk of the surgery, and reduces the patient's pain and medical costs.

(4) In this solution, the weights of respective convolutional kernels in the feature fusion network are adjusted based on the ray model to improve the ray consistency of the extracted features. This ensures that the impact of ray propagation is considered in the feature extraction process. The convolutional weight adjustment method can enhance the spatial interpretability of imaging data and ensure the accuracy of the feature extraction, thereby further improving the precision of 3D model reconstruction.

With the technical solution, augmented reality, ray modeling, and feature fusion networks are comprehensively applied to achieve real-time and accurate overlay presentation of the 3D model of the fracture model in the actual surgical scene. It significantly enhances diagnostic and surgical accuracy and efficiency, providing substantial clinical application value.

### BRIEF DESCRIPTION OF DRAWINGS

To more clearly illustrate the technical solutions in the embodiments of the present disclosure, the accompanying drawings required for the description of the embodiments are briefly introduced below. It is apparent that the drawings described below only pertain to some embodiments of the present disclosure. For those skilled in the art, other drawings may also be derived based on these illustrations without any inventive effort.

FIG. 1 illustrates an example flowchart of the method for overlay presentation of a skeletal image based on augmented reality according to an embodiment of the present disclosure;

FIG. 2 illustrates a flowchart of an example operation corresponding to the step S110 in FIG. 1;

FIG. 3 illustrates a flowchart of an example operation corresponding to the step S120 in FIG. 1;

FIG. 4 illustrates a flowchart of an example operation corresponding to step S150 in FIG. 1;

FIG. 5 illustrates an example structural block diagram of the system for overlay presentation of a skeletal image based on augmented reality according to an embodiment of the present disclosure; and

FIG. 6 is a schematic structural diagram of an embodiment of the electronic device of the present disclosure.

### DETAILED DESCRIPTION OF PREFERRED EMBODIMENTS

To make the objectives, technical solutions, and advantages of the embodiments of the present disclosure clearer, the technical solutions of the embodiments will be described clearly and completely below with reference to the accompanying drawings. It is obvious that the described embodiments represent only a portion of the embodiments of the present disclosure, and not all possible embodiments. All other embodiments obtained by those skilled in the art based on the described embodiments, without involving inventive efforts, shall fall within the scope of protection of the present disclosure.

Unless otherwise defined, the technical or scientific terms used in this present disclosure shall have the meanings generally understood by a person of ordinary skill in the relevant field. The terms “first,” “second,” and similar expressions used herein do not indicate any order, quantity, or importance, but are merely used to distinguish different components. Similarly, terms such as “a,” “an,” or “the” do not imply a limitation in quantity but rather indicate the presence of at least one. The terms “comprise,” “include,” and similar expressions are intended to mean that the elements or objects listed before such terms encompass those listed after them and their equivalents, without excluding other elements or objects. The terms “connect” or “couple” and similar expressions are not limited to physical or mechanical connections but may also include electrical connections, whether direct or indirect.

It should be noted that terms such as “upper,” “lower,” “left,” “right,” “front,” and “rear” used in this present disclosure are merely intended to describe relative positional relationships. These relationships may change accordingly if the absolute position of the described object is changed.

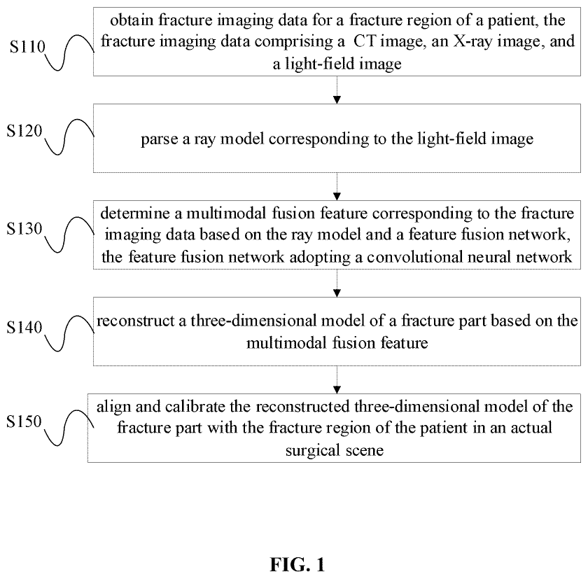

FIG. 1 illustrates an example flowchart of the method for overlay presentation of a skeletal image based on augmented reality according to an embodiment of the present disclosure.

As for the execution subject of the method in the embodiments of the present disclosure, it may be any controller or processor with computing or processing capabilities. It can be fully or partially integrated into AR glasses. By introducing light-field image and a feature fusion network, combined with the ray consistency constraint and convolution kernel weight adjustment, high-precision reconstruction of the 3D structure of the fracture part can be achieved, real-time visual guidance through can be provide AR technology, significantly enhancing the accuracy and efficiency of fracture diagnosis and treatment.

In some examples, the execution subject may be implemented in a server-side or client-side configuration through software, hardware, or a combination of both, without limitation.

In the following, the technical details of the present disclosure will be described using a virtual fracture overlay platform as an example execution subject. However, it should be understood that one or more of the steps involved in the process described below may be implemented by one or more controllers or software modules deployed on the client or server side.

As shown in FIG. 1, at block S110, fracture imaging data of the patient's fracture region is obtained. The fracture imaging data includes a CT image, an X-ray image, and a light-field image.

In some embodiments, the patient undergoes examination of the fracture region using various medical imaging devices. These devices upload the corresponding imaging data to a hospital service platform, and the AR glass retrieves the relevant imaging data from the platform based on the patient's ID. Here, the CT image may be obtained by scanning the patient's fracture region with a CT scanner to generate high-resolution cross-sectional images. The X-ray image may be captured using X-ray equipment to provide two-dimensional (2D) planar information of the fracture part. Additionally, a light-field camera may be used to capture the light-field image of the fracture region, recording information about the direction and intensity of light rays. Through the CT, the X-ray imaging, and the light-field camera, the fracture region is scanned from multiple angles and at multiple levels, thereby obtaining high-resolution 2D imaging data and light-field images.

Regarding the details of the acquisition process of the light-field camera, the light-field camera may be positioned appropriately to fully capture the ray propagation information in the fracture region. Then, camera parameters such as focal length and exposure time may be adjusted to obtain image data that includes information about the ray propagation path.

At block S120, the ray model corresponding to the light-field images is parsed.

In some embodiments, a ray tracing algorithm may be used to build a ray model based on the light data in the light-field image. The direction, intensity, and propagation path of each ray may be parsed, it provides spatial propagation information of the light in the patient's fracture region. This supports the understanding of the 3D structure and material characteristics of the fracture part.

At block S130, the multimodal fusion feature corresponding to the fracture imaging data is determined based on the ray model and a feature fusion network. The feature fusion network utilizes a convolutional neural network (CNN).

In some embodiments, the CNN is used to extract core features of the fracture part from the CT image and the X-ray image, and ray propagation characteristics are extracted from the ray model as additional features. Then, features from different modal image data are fused. Thus, the multimodal fusion feature integrates information from the CT image, the X-ray image, and the light-field image, enhancing the understanding and representation of the fracture region and providing rich input data for the reconstruction of the 3D model.

In certain examples of embodiments of the present disclosure, during the feature fusion process, the ray model may be utilized as guidance information, and a ray consistency constraint may be introduced into the feature fusion network to enable the consistency of the fused feature along the ray propagation paths. Specifically, the loss function L of the feature fusion network may be defined as:

$$L = {λ}_{ray} {L}_{ray} + {λ}_{feat} {L}_{feat} \qquad ( 1 )$$

$${L}_{ray} = {\sum }_{p ∈ P} {‖ {F}_{fusion} ( p ) - R ( p ) ‖}^{2} \qquad ( 2 )$$

$${L}_{feat} = {\sum }_{f ∈ F} {‖ {F}_{f u s i o n} ( f ) - {F}_{i n p u t} ( f ) ‖}^{2} \qquad ( 3 )$$

where L_{feat} represents a feature matching loss configured to enable a consistency between the fused feature and an input feature in a feature space; L_{ray} represents a ray consistency constraint loss configured to enable the consistency of the fused feature along the ray propagation path; λ_{ray} is a weight coefficient of the L_{ray}, λ_{feat} is a weight coefficient of the L_{feat}; p represents a point on the ray, P represents a set of all ray points, F_{fusion}(p) fusion represents a fused feature at point p, R(p) represents a feature value of the ray model at the point p; I represents a point on a feature map, F represents a set of all points on the feature map, F_{fusion}(f) represents a fused feature at the point I, and Fᵢₙₚᵤₜ(f) represents an input feature at the point f.

Here, F_{fusion} represents the unified fused feature of the CT image, the X-ray image, and the light-field image. As an example, deep convolutional neural networks (CNNs) may be used to extract features from the CT image, the X-ray image, and the light-field image respectively. For each imaging modality, the multi-scale feature map may be extracted. The extracted feature may be represented as follows:

$${F}_{C T} = {CNN}_{C T} ( {I}_{C T} ) \qquad ( 4 )$$

$${F}_{X - ray} = {CNN}_{X - ray} ( {I}_{X - ray} ) \qquad ( 5 )$$

$${F}_{L F} = {CNN}_{L F} ( {I}_{L F} ) \qquad ( 6 )$$

where I_{CT}, I_{X-ray}, and I_{LF} represent the CT image, X-ray image, and the light-field image respectively; F_{CT}, F_{X-ray}, and F_{X-ray} represent the corresponding feature map.

The feature maps of the CT image, the X-ray image, and the light-field image may be aligned by using the image registration technology, ensuring consistency of features from different modalities. The registered features may be represented as follows:

$${F}_{{CT}^{′}} = Align ( {F}_{C T} , {F}_{X - r a y} ) \qquad ( 7 )$$

$${F}_{{LF}^{′}} = Align ( {F}_{L F} , {F}_{X - r a y} ) \qquad ( 8 )$$

Then, the aligned multimodal features may be concatenated to obtain the fused feature:

$${F}_{fusion} = Fusion ( {F}_{{CT}^{′}} , {F}_{X - ray} , {F}_{{LF}^{′}} ) \qquad ( 9 )$$

Furthermore, based on the ray model, weights of respective convolutional kernels in the feature fusion network may be adjusted to enhance the ray consistency of features extracted by convolution:

$${F}_{c o n v} = σ ( {\sum }_{k = 1}^{K} {W}_{c o n v}^{( k )} * {F}_{input}^{( k )} + {b}^{( k )} ) \qquad ( 10 )$$

$${W}_{c o n v}^{( k )} = {W}_{b a s e}^{( k )} + α {R}_{c o n v}^{( k )} \qquad ( 11 )$$

$${R}_{c o n v}^{( k )} = {\sum }_{i = 1}^{N} {w}_{i} {R}_{i}^{( k )} \qquad ( 12 )$$

$${w}_{i} = \frac{1}{Z} exp ( - \frac{1}{{θ}^{2}} {\sum }_{p ∈ {P}_{i}} {‖ {F}_{fusion} ( p ) - {R}_{i} ( p ) ‖}^{2} ) \qquad ( 13 )$$

wherein F_{conv} represents a feature map after convolution,

$${W}_{c o n v}^{( k )}$$

and b^{(k)} respectively represent a weight and a bias of the k-th convolution kernel,

$${F}_{input}^{( k )}$$

represents k-th channel of an input feature map, * represents a convolution operation, and σ represents a ReLU activation function;

$${W}_{b a s e}^{( k )}$$

represents a base weight of the k-th convolution kernel; α represents an adjustment coefficient configured to control an extent of influence of the ray model on a convolution kernel weight;

$${R}_{c o n v}^{( k )}$$

represents an adjusted weight guided by the ray model for the k-th convolution kernel; wᵢ represents a weight of i-th ray, Pᵢ represents a set of all points on the i-th ray,

$${R}_{i}^{( k )}$$

represents a feature value of the i-th ray in the k-th convolution kernel, and N represents a number of rays; Rᵢ(p) represents a feature value of the i-th ray at a point p, θ represents a hyperparameter for controlling a weight distribution, and Z represents a normalization factor.

Here, the weight wᵢ may reflect the influence of each ray on the fused feature, which comprehensively considers the importance of the ray propagation path and the alignment with features of other modalities. During convolution, the weights of the convolution kernels are adjusted using the ray model, ensuring that the reconstructed features have higher ray consistency.

Here, the use of the CT image, the X-ray image and the light field image for multi-modal data fusion may effectively combine the advantages of different imaging modalities, contributing to high-precision 3D reconstruction. Notably, the ray tracing technology can provide rich ray propagation information, enhancing the depth perception and realism of the 3D reconstruction.

In this embodiment, feature fusion is performed by introducing the ray model, ensuring consistency and coherence of the multimodal feature along the ray propagation path. Specifically, the ray consistency constraint loss function is utilized to ensure consistency of the fused feature in the ray propagation path. Additionally, the weight of the convolutional kernel is adjusted under the guidance of the ray model, enabling different kernels to capture diverse feature information, and enhancing the variety and accuracy of the feature extraction.

At block S140, a 3D model of the fracture part is reconstructed based on the multimodal fusion feature.

In some embodiments, a voxel generation network is used to generate a 3D voxel model based on the fused feature. Specifically, the voxel generation network may include convolutional layers and transposed convolutional layers. Higher-level features (including spatial information and depth information) are extracted through a plurality of convolutional layers. The higher-level features are converted into the 3D voxel model through the transposed convolutional layers, and the features are mapped to the 3D space by expanding the spatial dimension of the feature map. Subsequently, a triangular mesh generation algorithm, such as the marching cubes algorithm, may be applied to extract the 3D surface model of the fracture part from the voxel model.

In some examples of embodiments of the present disclosure, the multimodal fusion features are input into a 3D CNN to generate an initial 3D model of the fracture part. The initial model may be then input into a generative adversarial network (GAN) to update it, to obtain the final 3D fracture model. Specifically, the generative adversarial network includes a generator and a discriminator.

The generator receives the initial 3D model of the fracture part as input, and generates a detail-enhanced 3D model through multiple layers of convolution and deconvolution operations. Within the convolutional layers of the generator, a channel attention module and a spatial attention module are introduced to determine the importance of each position and adjust the weight of the feature map accordingly. The formula is as follows:

$${F}_{C A} = Sigmoid ( FC 2 ( R e L U ( FC 1 ( GAP ( F ) ) ) ) ) \qquad ( 14 )$$

$${F}_{S A} = Sigmoid ( Conv ( Concat [ AvgPool ( F ) ; MaxPool ( F ) ] ) ) \qquad ( 15 )$$

where F_{CA} represents a channel attention map, FC1 and FC2 are fully connected layers, and GAP is global average pooling; F_{SA} represents a spatial attention map; AvgPool and MaxPool are an average pooling operation and a max pooling operation respectively, and Concat denotes a feature concatenation operation;

The discriminator receives the real 3D model and the generated detail-enhanced 3D model, and determines the authenticity of the input through multi-layer convolution operations.

The loss function L_{G} of the generator may be defined as:

$${L}_{G} = {L}_{g e n} + {λ}_{pixel} {L}_{pixel} \qquad ( 16 )$$

$${L}_{g e n} = - log ( {D}_{a t t n} ( {G}_{a t t n} ( {V}_{i nitial} ) ) ) \qquad ( 17 )$$

$${L}_{pixel} = {‖ {V}_{real} - {G}_{attm} ( {V}_{i nitial} ) ‖}_{1} \qquad ( 18 )$$

where L_{gen} is a generator loss, Dₐₜₜₙ denotes a discriminator network, Gₐₜₜₙ denotes a generator network, and Vᵢₙᵢₜᵢₐₗ represents the initial 3D model of the fracture part; Lₚᵢₓₑₗ is a pixel-level reconstruction loss; ∥·∥₁ represents a L1 norm; Vᵣₑₐₗ is a real 3D model of the fracture part; and λₚᵢₓₑₗ is a weight coefficient of the pixel-level reconstruction loss.

The pixel-level reconstruction loss may ensure that the generated 3D model is consistent with the real model in terms of details.

The loss function L_{D} of the discriminator is defined as:

$${L}_{D} = - log ( {D}_{a t t n} ( {V}_{real} ) ) - log ( 1 - {D}_{a t t n} ( {G}_{a t t n} ( {V}_{i nitial} ) ) ) \qquad ( 19 )$$

In this embodiment, a 3D convolutional neural network (3D CNN) may be used to generate the initial 3D model of the fracture region from the multimodal fusion features. A generator and a discriminator including attention mechanisms are then constructed to generate the detail-enhanced 3D model through multiple layers of convolution and deconvolution operations. Here, the channel attention module and the spatial attention module are introduced into the convolutional layer of the generator to determine the importance of each position and adjust the weight of the feature map accordingly. The discriminator receives the real 3D model and the generated detail-enhanced model, and determines the authenticity of the input through multi-layer convolutional processing.

Thus, by using the 3D CNN to generate the initial 3D model, it can effectively capture of spatial structure information of the fracture region and improve the basic reconstruction accuracy of the model. By introducing the channel attention module and the spatial attention module in the generator's convolutional layers, the importance of each position may be determined, and the weight of the feature map may be adjusted, allowing the generator to pay more attention to the details of critical regions. Through adversarial training, the discriminator may continuously improve the authenticity of the generated model, enabling the generated 3D model more realistic in details. Furthermore, in the design of the loss function, a pixel-level reconstruction loss is introduced based on the adversarial loss to guide the generator to generate the more realistic 3D model, ensuring that the generated 3D model is consistent with the real model in details.

At block S150, the reconstructed 3D model of the fracture region is aligned and calibrated with the patient's fracture region in the actual surgical scene.

In some embodiments, AR glasses may align and calibrate the information collected by the visual sensing module with the 3D model of the fracture part. For example, by combining data from the camera and inertial sensors, the pose of the AR glasses may be calculated in real time, ensuring precise alignment of the virtual image and the real scene. For example, through feature point detection and tracking algorithms, feature points in the surgical scene may be identified and tracked in real time, ensuring that the virtual image can dynamically adapt to changes in the surgical scene. Thus, through the high-precision 3D reconstruction model and real-time feature fusion, the AR system can accurately overlay the virtual 3D fracture model on the real surgical scene.

Through the embodiment of the present disclosure, high-precision 3D reconstruction and accurate AR overlay are provided, enabling doctors to comprehensively and intuitively view the 3D structure of the fracture part. With multimodal feature fusion and ray model guidance, high precision 3D reconstruction model may be provided to help doctors make more informed and precise judgments about the fracture. In addition, through AR technology, doctors can intuitively present the fracture condition and treatment plan to the patient, enhancing the patient's understanding and trust.

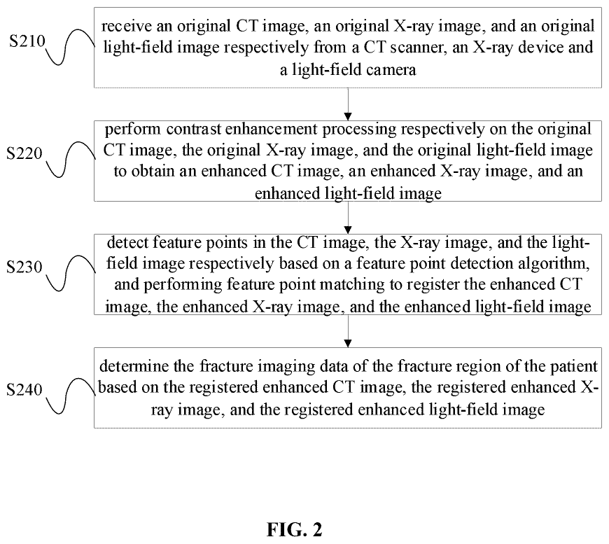

FIG. 2 illustrates an example operation flow corresponding to Step S110 in FIG. 1.

As shown in FIG. 2, at block S210, an original CT image, an original X-ray image, and an original light-field image are received from a CT scanner, an X-ray device, and a light-field camera, respectively.

At block S220, contrast enhancement is performed respectively on the original CT image, the original X-ray image, and the original light-field image to obtain an enhanced CT image, an enhanced X-ray image, and an enhanced light-field image.

Specifically, contrast limited adaptive histogram equalization (CLAHE) may be applied to process the original CT image. The image is divided into multiple small blocks, histogram equalization is performed on each block, but the contrast of each gray level (clipping limit) is restricted. Then, the processed small blocks are recombined, and bilinear interpolation is performed to eliminate boundary effects. The specific formula is as follows:

$${I}_{CLAHE} ( x , y ) = \frac{L - 1}{M N} {\sum }_{k = 0}^{{I}_{1} ( x , y )} {h}_{clip} ( k ) \qquad ( 20 )$$

where I₁(x, y) represents a pixel value of the original CT image at a position (x, y), I_{CLAHE}(x, y) represents a pixel value of the enhanced CT image at the corresponding position; L is a number of grayscale levels; M and N are width and height of an image respectively, h_{clip}(k) is a cumulative distribution function of a clipped histogram;

Here, CLAHE can significantly enhance the contrast of the fracture edge, effectively improving contrast while avoiding over-enhancement. This enables the subtle fracture line more clearly visible, which helps to enhance the clarity of structural contours in the reconstructed 3D model.

The original X-ray image is process by using the adaptive contrast enhancement (ACE). Specifically, the contrast is adaptively adjusted based on the local brightness characteristics of the image. A local contrast enhancement factor is then determined for each pixel. The specific formula is as follows:

$${I}_{adaptive} ( x , y ) = {I}_{2} ( x , y ) \cdot ( 1 + \frac{{I}_{2} ( x , y ) − {μ}_{local} ( x , y )}{{σ}_{local} ( x , y ) + {o}^{`}} ) \qquad ( 21 )$$

where I₂(x,y) represents a pixel value of the original X-ray image at the position (x,y), I_{adaptive}(x,y) is a pixel value of the enhanced X-ray image at a corresponding position; μ_{local}(x,y) is a mean value of images at a local neighborhood of the position (x,y), σ_{local}(x,y) is a standard deviation of images in the local neighborhood of the position (x,y), and Ò represents a preset constant;

Here, in the fracture image, adaptive contrast enhancement can enhance the contrast in local regions, highlight the details of the fracture region. This helps ensure that the reconstructed model more prominently highlights the structure and location of key fracture parts.

The original light-field image is processed by using a multi-scale Retinex algorithm. Specifically, by simulating the human visual system's ability to adapt to changes in illumination, the contrast of the light-field image is enhanced, and a weighted sum across different scales is determined. The specific formula is as follows:

$${I}_{R e t i n e x} ( x , y ) = {\sum }_{s} w ( s ) ( log {I}_{3} ( x , y ) - log ( {G}_{s} * I ) ( x , y ) ) \qquad ( 22 )$$

where I₃(x,y) represents a pixel value of the original light-field image at the position (x,y), I_{adaptive}(x,y) is a pixel value of the enhanced light-field image at the corresponding position; w(s) represents a weight corresponding to scale s, and Gₛ denotes a Gaussian filter with the scale s;

Here, the multi-scale Retinex algorithm can effectively enhance the contrast of the light-field image by simulating the human visual system's adaptability to varying lighting conditions. This enhances the contrast of the light-field image, improves detail visibility under different illumination environments, contributing to greater diagnostic accuracy.

At block S230, feature points in the CT image, the X-ray image, and the light-field image are detected respectively based on a feature point detection algorithm, and feature point matching is performed to register the enhanced CT image, the enhanced X-ray image, and the enhanced light-field image.

In some embodiments, feature point detection algorithms such as scale-invariant feature transform (SIFT), speeded-up robust feature (SURF), or oriented FAST and rotated BRIEF (ORB) may be used to detect feature points in each enhanced modality image. Feature point matching is then performed to achieve image alignment and registration.

At block S240, the fracture imaging data of the fracture region of the patient is determined based on the registered enhanced CT image, the registered enhanced X-ray image, and the registered enhanced light-field image.

In this embodiment, through contrast enhancement processing, fracture details become more visible in the image, providing more detailed information for 3D reconstruction, and improving the accuracy and realism of the reconstructed result. Additionally, the use of feature point registration can ensure spatial alignment across different imaging modalities, enabling full use of multimodal data in the reconstruction process, thereby improving the precision of the reconstructed model.

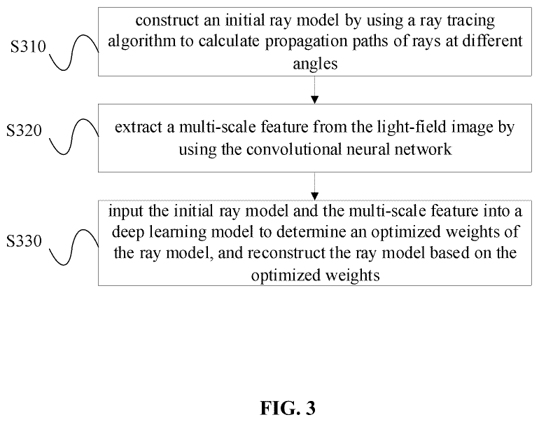

FIG. 3 illustrates an example operation flow corresponding to step S120 in FIG. 1.

At block S310, an initial ray model is constructed by using a ray tracing algorithm to calculate propagation paths of rays at different angles.

Specifically, high-resolution light-field images may be obtained from the light-field camera. The light-field images is subjected to denoising and contrast enhancement. A ray tracing algorithm is applied to determine the propagate paths of the ray at various angles, to obtain the initial ray model. The specific formula is as follows:

$${R}_{i nitial} ( x , y , θ , ϕ ) = {\sum }_{e = 1}^{N} {w}_{e} \cdot L ( {x}_{e} , {y}_{e} , θ , ϕ ) \qquad ( 23 )$$

where Rᵢₙᵢₜᵢₐₗ(x,y,θ,ϕ) represents the initial ray model at a position (x,y) and an angle (θ,ϕ), N denotes a number of rays; wₑ represents a weight of the e-th ray, and L(xₑ,yₑ,θ,ϕ) represents a ray feature of the e-th ray at a position (xₑ,yₑ);

Here, the propagation paths of rays at different angles are determined using the ray tracing algorithm, direction and intensity of the ray are accurately determined to construct the initial ray model, ensuring the basic accuracy of the ray model.

At block S320, a multi-scale feature is extracted from the light-field image by using the CNN.

Here, the multi-scale feature is extracted from the light-field image through the CNN via multiple layers of convolution and pooling operations, ensuring the acquisition of rich feature information, and providing strong support for the optimization of the ray model.

$${F}_{L F} = {CNN}_{L F} ( {I}_{L F} ) \qquad ( 24 )$$

where I_{LF} represents an input light-field image, and F_{LF} denotes the extracted multi-scale feature;

At block S330, the initial ray model and the multi-scale feature are input into a deep learning model to determine an optimized weights ŵₑ of the ray model, and the ray model is reconstructed based on the optimized weights ŵₑ.

Here, to improve the accuracy and consistency of the initial ray model, a deep learning model is adopted to the weights in the initial model by learning the relationship between the features of the light-field image and the ray model. For example, the deep learning model includes multiple convolutional layers and fully connected layers. The input layer receives the initial ray model and the extracted feature maps, the hidden layer extracts high-level features through convolution operations, and perform weight optimization through the fully connected layer. The specific formula is as follows:

$${\hat{w}}_{e} = DNN ( {R}_{i nitial} ( x , y , θ , ϕ ) , {F}_{L F} ) , \qquad ( 25 )$$

$${R}_{t a r g e t} ( x , y , θ , ϕ ) = {\sum }_{e = 1}^{N} {\hat{w}}_{e} \cdot L ( {x}_{e} , {y}_{e} , θ , ϕ ) , \qquad ( 26 )$$

where DNN(⋅) represents a deep neural network function, R_{target}(x,y,θ,ϕ) represents the reconstructed ray model, and ŵₑ denotes a weight optimized by the deep learning model.

In this embodiment, by inputting the initial ray model and multi-scale features into the deep learning model, the relationship between the feature of the light-field image and the ray model is learned to determine the optimized weight of the ray model. The weight is then used to reconstruct the ray model, thereby significantly improving the accuracy and consistency of the model. Thus, through the optimized ray model, the accuracy of the ray propagation paths is ensured, enabling the reconstructed 3D model to more accurately reflect detailed information in the fracture region and improving the precision and coherence of the 3D reconstruction.

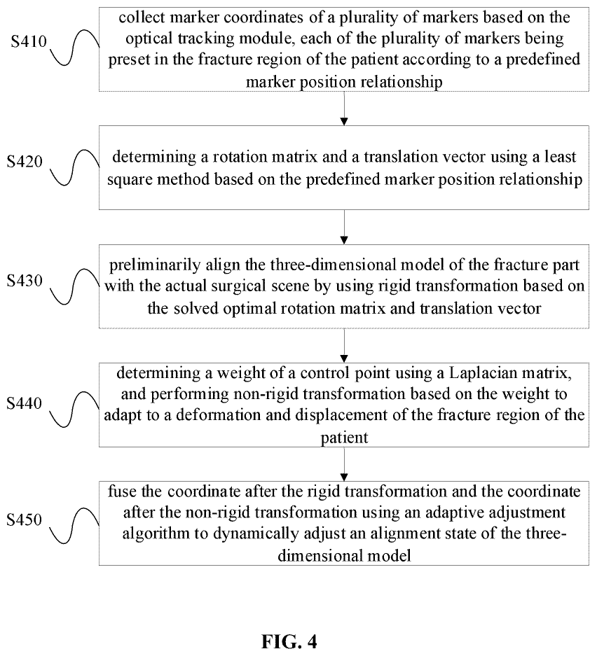

FIG. 4 illustrates an example operation flow corresponding to step S150 in FIG. 1.

As shown in FIG. 4, at block S410, marker coordinates of a plurality of markers are collected based on the optical tracking module, each of the plurality of markers is preset in the fracture region of the patient according to a predefined marker position relationship.

In some embodiments, markers are placed on the patient's fracture region and surgical instruments, and then 3D coordinates of these markers may be collected in real time through the optical tracking module in the VR glass. Through the optical tracking module, coordinates of multiple markers preset in the patient's fracture region may be accurately collected, ensuring high-precision and reliability of the data.

At block S420, a rotation matrix R and a translation vector t are determined using a least square method based on the predefined marker position relationship. The specific formula is as follows:

$${min}_{R , t} {\sum }_{g} {‖ R \cdot {H}_{g} + t - {Q}_{g} ‖}^{2} \qquad ( 27 )$$

Optimal R and t are solved through a singular value decomposition, where H_{g} and Q_{g} respectively represent coordinates of g-th marker in the three-dimensional model of the fracture part and the actual surgical scene.

At block S430, the three-dimensional model of the fracture part is preliminarily aligned with the actual surgical scene by using rigid transformation based on the optimal R and t. The specific formula is as follows:

$${T}_{rigid} ( x ) = R \cdot x + t \qquad ( 28 )$$

where x represents an initial coordinate of any voxel point in the 3D model of the fracture part, and T_{rigid}(x) represents a coordinate after rigid transformation.

Here, based on the position relationship of the markers, the least squares method is used to determine the rotation matrix and translation vector, and the optimal solution is obtained through singular value decomposition (SVD) to achieve preliminary alignment of the 3D model.

At block S440, a weight wᵤ of a control point pᵤ are determined using a Laplacian matrix, and non-rigid transformation is performed based on the weight wᵤ to adapt to a deformation and displacement of the fracture region of the patient. The specific formula is as follows:

$${T}_{non - rigid} = x + {{\sum }_{u = 1}}^{N} {w}_{u} ϕ ( ‖ x - {p}_{u} ‖ ) \qquad ( 29 )$$

where T_{non-rigid}(x) represents a coordinate after non-rigid transformation, and ϕ(r) denotes a Gaussian function.

Here, the Laplacian matrix is used to compute the weight of each control point, and the non-rigid transformation is performed based on these weights to accommodate deformation and displacement in the patient's fracture region. Thus, initial alignment is achieved through rigid transformation, followed by fine adjustment via non-rigid transformation, thereby improving alignment accuracy.

At block S450, the coordinate T_{rigid}(x) after the rigid transformation and the coordinate T_{non-rigid}(x) after the non-rigid transformation are fused using an adaptive adjustment algorithm to dynamically adjust an alignment state of the three-dimensional model. The specific formula is as follows:

$${T}_{adaptive} ( x ) - {T}_{rigid} ( x ) + β \cdot ( {T}_{non - rigid} ( x ) + {T}_{rigid} ( x ) ) \qquad ( 30 )$$

where T_{adaptive}(x) represents a coordinate after the adaptive adjustment, and β denotes an adaptive adjustment coefficient.

Thus, through the adaptive adjustment algorithm, the coordinates after the rigid transformation and non-rigid transformation are fused to dynamically update the alignment state of the 3D model. In this embodiment, the position data of the patient and surgical instruments is collected in real-time through the optical tracking module, the alignment state of the 3D model is dynamically updated. This ensures high consistency between the model and the actual surgical scene during the operation. As a result, the doctors can directly refer to the virtual surgical plan (e.g., surgical navigation path) in the 3D model during the operation, making the surgical process more efficient and accurate.

It should be noted that, for the sake of clarity, the foregoing method embodiments have been described as a series of sequential actions. However, those skilled in the art will understand that the present disclosure is not limited to the described sequence, and that certain steps may be performed in a different order or concurrently, depending on implementation needs. Furthermore, it should be understood by those skilled in the art that the embodiments described in the specification are preferred embodiments, and the actions and modules involved are not necessarily essential to the present disclosure. Each embodiment focuses on different aspects; where a specific detail is not fully elaborated in one embodiment, reference can be made to the relevant descriptions in other embodiments.

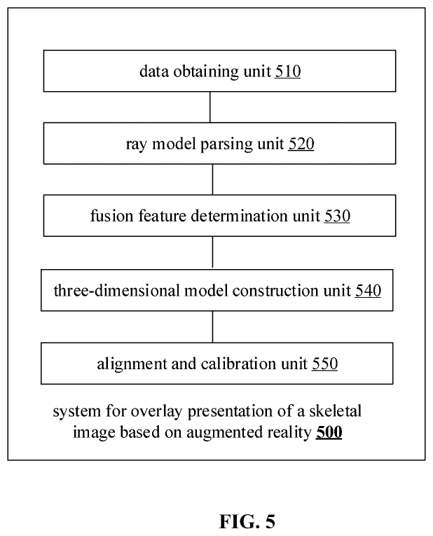

FIG. 5 illustrates an example structural block diagram of a system for overlay presentation of a skeletal image based on augmented reality according to an embodiment of the present disclosure.

As shown in FIG. 5, the system 500 includes a data obtaining unit 510, a ray model parsing unit 520, a fusion feature determination unit 530, a 3D model construction unit 540 and an alignment and calibration unit 550.

The data obtaining unit 510 is configured to obtain fracture imaging data for a fracture region of a patient, the fracture imaging data including a CT image, an X-ray image, and a light-field image.

The ray model parsing unit 520 is configured to parse a ray model corresponding to the light-field image, the ray model providing spatial propagation information of a light in the fracture region of the patient.

The fusion feature determination unit 530 is configured to determine a multimodal fusion feature corresponding to the fracture imaging data based on the ray model and a feature fusion network, the feature fusion network adopting a convolutional neural network.

During the feature fusion process, the ray model is utilized as guidance information, and a ray consistency constraint is introduced into the feature fusion network to ensure a consistency of a fused feature along a ray propagation path. The loss function L of the feature fusion network is defined as follows:

$$L = {λ}_{r a y} {L}_{r a y} + {λ}_{feat} {L}_{feat}$$

$${L}_{r a y} = {\sum }_{p ∈ P} {‖ {F}_{fusion} ( p ) - R ( p ) ‖}^{2}$$

$${L}_{feat} = {\sum }_{f ∈ F} {‖ {F}_{fusion} ( f ) - {F}_{input} ( f ) ‖}^{2}$$

where L_{feat} represents a feature matching loss configured to enable a consistency between the fused feature and an input feature in a feature space; L_{ray} represents a ray consistency constraint loss configured to enable the consistency of the fused feature along the ray propagation path; λ_{ray} is a weight coefficient of the L_{ray}, λ_{feat} is a weight coefficient of the L_{feat}; p represents a point on the ray, P represents a set of all ray points, F_{fusion}(p) represents a fused feature at point p, R(p) represents a feature value of the ray model at the point p; f represents a point on a feature map, F represents a set of all points on the feature map, F_{fusion}(f) represents a fused feature at the point I, and Fᵢₙₚᵤₜ(f) represents an input feature at the point f.

The weights of respective convolution kernels in the feature fusion network are adjusted based on the ray model to enhance a ray consistency of features extracted by convolution:

$${F}_{c o n v} = σ ( {\sum }_{k = 1}^{K} {W}_{c o n v}^{( k )} * {F}_{i n p u t}^{( k )} + {b}^{( k )} )$$

$${W}_{c o n v}^{( k )} = {W}_{b a s e}^{( k )} + α {R}_{c o n v}^{( k )}$$

$${R}_{c o n v}^{( k )} = {\sum }_{i = 1}^{N} {w}_{j} {R}_{i}^{( k )}$$

$${w}_{i} = \frac{1}{Z} exp ( - \frac{1}{{θ}^{2}} {\sum }_{p ∈ P} {‖ {F}_{fusion} ( p ) - {R}_{i} ( p ) ‖}^{2} )$$

where F_{conv} represents a feature map after convolution,

$${W}_{c o n v}^{( k )}$$

and b^{(k)} respectively represent a weight and a bias of k-th convolution kernel,

$${F}_{input}^{( k )}$$

represents k-th channel of an input feature map, * represents a convolution operation, and σ represents a ReLU activation function;

$${W}_{b a s e}^{( k )}$$

represents a base weight of the k-th convolution kernel; α represents an adjustment coefficient configured to control an extent of influence of the ray model on a convolution kernel weight;

$${R}_{c o n v}^{( k )}$$

represents an adjusted weight guided by the ray model for the k-th convolution kernel; wᵢ represents a weight of i-th ray, Pᵢ represents a set of all points on the i-th ray,

$${R}_{i}^{( k )}$$

represents a feature value of the i-th ray in the k-th convolution kernel, and N represents a number of rays; Rᵢ(p) represents a feature value of the i-th ray at a point p, θ represents a hyperparameter for controlling a weight distribution, and Z represents a normalization factor.

The three-dimensional model construction unit 540 is configured to reconstruct a three-dimensional model of a fracture part based on the multimodal fusion feature.

The alignment and calibration unit 550 is configured to align and calibrate the reconstructed three-dimensional model of the fracture part with the fracture region of the patient in an actual surgical scene.

In some embodiments, the present disclosure further provides a non-transitory computer-readable storage medium, in which one or more programs including executable instructions are stored. These instructions can be read and executed by an electronic device (including but not limited to a computer, server, or network device) to perform the method for overlay presentation of a skeletal image based on augmented reality as described above.

In some embodiments, the present disclosure also provides a computer program product, which includes a computer program stored on a non-transitory computer-readable storage medium. The computer program includes program instructions that, when executed by a computer, enable the computer to perform the method for overlay presentation of a skeletal image based on augmented reality as described above.

In some embodiments, the present disclosure also provides an electronic device, which includes at least one processor; and a memory communicatively connected to the at least one processor. The memory stores instructions that can be executed by the at least one processor, and when executed, cause the processor to perform the method for overlay presentation of a skeletal image based on augmented reality.

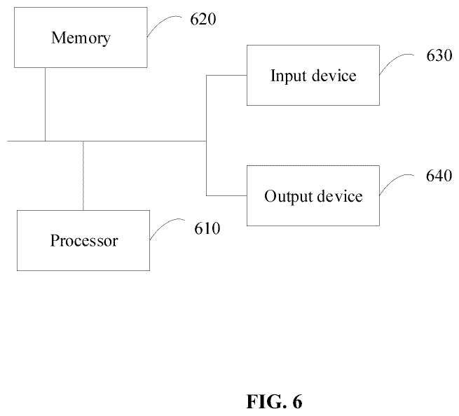

FIG. 6 is a schematic diagram of the hardware structure of an electronic device for executing the method for overlay presentation of a skeletal image based on augmented reality according to a further embodiment of the present disclosure. As shown in FIG. 6, the device includes one or more processors 610 and a memory 620 (FIG. 6 illustrates a single processor 610 as an example).

The device for executing the method for overlay presentation of a skeletal image based on augmented reality may further include an input device 630 and an output device 640.

The processor 610, memory 620, input device 630, and output device 640 may be connected via a bus or other communication means. In FIG. 6, a bus connection is used as an example.

The memory 620, as a non-volatile computer-readable storage medium, may be used to store non-volatile software programs, computer-executable instructions, and modules, such as the program instructions/modules corresponding to the skeletal image overlay presentation method based on augmented reality technology described in the embodiments of this present disclosure. The processor 610 executes various functional present disclosures and data processing of the system by running the software programs, instructions, and modules stored in memory 620, thereby implementing the skeletal image overlay presentation method based on AR technology described above.

The memory 620 may include a program storage area and a data storage area, where: The program storage area can store the operating system and one or more present disclosure programs required for functionalities; The data storage area can store data created during device operation, among others. Additionally, the memory 620 may include high-speed random access memory (RAM) and non-volatile memory such as at least one disk storage device, flash memory device, or other types of non-volatile solid-state storage. In some embodiments, the memory 620 may optionally include memory remotely located from the processor 610. These remote memory units can be connected to the device via a network. Examples of such networks include, but are not limited to, the Internet, intranets, local area networks (LANs), mobile communication networks, or combinations thereof.

The input device 630 may be used to receive input in the form of numerical or character information, and to generate signals related to user settings and functional controls of the electronic device. The output device 640 may include a display screen or other display components.

One or more modules are stored in the memory 620, and when executed by one or more processors 610, cause the method for overlay presentation of a skeletal image based on augmented reality as described in any of the method embodiments above to be performed.

The aforementioned product is capable of executing the method provided in the embodiments of this present disclosure and includes the corresponding functional modules and beneficial effects. Technical details not fully described in this embodiment can be found in the method embodiments of the present disclosure.

The electronic device described in this embodiment may take various forms, including but not limited to:

Mobile communication devices: these devices feature mobile communication capabilities and are primarily intended for voice and data communication. Examples include smartphones, multimedia phones, feature phones, basic phones and so on.

Ultra-mobile personal computing devices: these belong to the category of personal computers with computing and processing capabilities, generally also featuring mobile internet access. Examples include PDAs (personal digital assistants), MIDs (mobile internet devices), UMPCs (ultra-mobile PCs) and so on.

Portable entertainment devices: devices capable of displaying and playing multimedia content. Examples include audio and video players, handheld gaming consoles, E-book readers, smart toys, portable in-car navigation systems.

Other onboard electronic devices with data interaction functionality, such as vehicle-mounted systems installed in cars.

The device embodiments described above are merely illustrative. The units described as separate components may or may not be physically separated, and the components displayed as units may or may not be physical entities. That is, they may reside in a single location or be distributed across multiple network nodes. Portions or all of the modules may be selected as needed to achieve the objectives of the embodiment.

Based on the descriptions provided above, it is clear to those skilled in the art that the various embodiments can be implemented by means of software combined with a general-purpose hardware platform, or alternatively, fully through hardware. With this understanding, the core of the technical solutions, or the part that contributes to the prior art, can essentially be embodied in the form of a software product. This computer software product may be stored on a computer-readable storage medium such as ROM, RAM, magnetic disk, optical disk, etc., and include a set of instructions that enables a computer device (such as a personal computer, server, or network device) to execute the methods described in the various embodiments or portions thereof.

Finally, it should be noted that the above embodiments are intended to illustrate the technical solutions of this present disclosure, and not to limit them. Although the present disclosure has been described in detail with reference to the foregoing embodiments, those of ordinary skill in the art will understand that modifications may still be made to the described technical solutions, or equivalent substitutions may be made for some of the technical features. Such modifications or substitutions shall not be considered to depart from the essence, spirit, or scope of the technical solutions of the embodiments of this present disclosure.

What is claimed is:

1. A method for overlay presentation of a skeletal image based on augmented reality (AR), applied to an AR glass, comprising:
obtaining fracture imaging data for a fracture region of a patient, the fracture imaging data comprising a computed tomography, a computed tomography (CT) image, an X-ray image, and a light-field image;
parsing a ray model corresponding to the light-field image, the ray model providing spatial propagation information of a light in the fracture region of the patient;
determining a multimodal fusion feature corresponding to the fracture imaging data based on the ray model and a feature fusion network, the feature fusion network adopting a convolutional neural network;
reconstructing a three-dimensional model of a fracture part based on the multimodal fusion feature; and
aligning and calibrating the reconstructed three-dimensional model of the fracture part with the fracture region of the patient in an actual surgical scene;
wherein the parsing the ray model corresponding to the light-field image comprises:
constructing an initial ray model by using a ray tracing algorithm to calculate propagation paths of rays at different angles:
$${R}_{i nitial} ( x , y , {θ}_{t} , ϕ ) = {\sum }_{e = 1}^{N} {w}_{e} \cdot L ( {x}_{e} , {y}_{e} , {θ}_{t} , ϕ )$$
wherein Rᵢₙᵢₜᵢₐₗ(x,y,θ,ϕ) represents the initial ray model at a position (x,y) and an angle (θ,ϕ), N denotes a number of rays; wₑ represents a weight of the e-th ray, and L(xₑ,yₑ,θ,ϕ) represents a ray feature of the e-th ray at a position (xₑ,yₑ);
extracting a multi-scale feature from the light-field image by using the convolutional neural network:
$${F}_{L F} = {CNN}_{L F} ( {I}_{L F} )$$
wherein I_{LF} represents an input light-field image, and F_{LF} denotes the extracted multi-scale feature;
inputting the initial ray model and the multi-scale feature into a deep learning model to determine an optimized weights ŵₑ of the ray model, and reconstructing the ray model based on the optimized weights ŵₑ:
$${\hat{w}}_{e} = DNN ( {R}_{i nitial} ( x , y , θ , ϕ ) , {F}_{L F} )$$
$${R}_{t a r g e t} ( x , y , {θ}_{t} , ϕ ) = {\sum }_{e = 1}^{N} {\hat{w}}_{e} \cdot L ( {x}_{e} , {y}_{e} , {θ}_{t} , ϕ )$$
wherein DNN(⋅) represents a deep neural network function, R_{target}(x,y,θ,ϕ) represents the reconstructed ray model, and ŵₑ denotes a weight optimized by the deep learning model;
wherein during a feature fusion process, the ray model is utilized as guidance information, and a ray consistency constraint is introduced into the feature fusion network to enable a consistency of a fused feature along a ray propagation path, a loss function of the feature fusion network being defined as:
$$L = {λ}_{ray} {L}_{ray} + {λ}_{feat} {L}_{feat}$$
$${L}_{ray} = {\sum }_{p ∈ P} {‖ {F}_{fusion} ( p ) - R ( p ) ‖}^{2}$$
$${L}_{feat} = {\sum }_{f ∈ F} {‖ {F}_{f u s i o n} ( f ) - {F}_{input} ( f ) ‖}^{2}$$
wherein L_{feat} represents a feature matching loss configured to enable a consistency between the fused feature and an input feature in a feature space; L_{ray} represents a ray consistency constraint loss configured to enable the consistency of the fused feature along the ray propagation path; λ_{ray} is a weight coefficient of the L_{ray}, λ_{feat} is a weight coefficient of the L_{feat}; p represents a point on the ray, P represents a set of all ray points, F_{fusion}(p) represents a fused feature at point p, R(p) represents a feature value of the ray model at the point p; f represents a point on a feature map, F represents a set of all points on the feature map, F_{fusion}(f) represents a fused feature at the point f, and Fᵢₙₚᵤₜ(f) represents an input feature at the point f.

2. The method of claim 1, wherein weights of respective convolution kernels in the feature fusion network are adjusted based on the ray model to enhance a ray consistency of features extracted by convolution:
$${F}_{c o n v} = σ ( {\sum }_{k = 1}^{K} {W}_{c o n v}^{( k )} * {F}_{input}^{( k )} + {b}^{( k )} )$$
$${W}_{c o n v}^{( k )} = {W}_{b a s e}^{( k )} + α {R}_{c o n v}^{( k )}$$
$${R}_{c o n v}^{( k )} = {\sum }_{i = 1}^{N} {w}_{i} {R}_{i}^{( k )}$$
$${w}_{i} = \frac{1}{Z} exp ( - \frac{1}{{θ}^{2}} {\sum }_{p ∈ {P}_{i}} {‖ {F}_{f u s i o n} ( p ) - {R}_{i} ( p ) ‖}^{2} )$$
wherein F_{conv} represents a feature map after convolution,
$${W}_{conv}^{( k )}$$
and b^{(k)} respectively represent a weight and a bias of k-th convolution kernel,
$${F}_{input}^{( k )}$$
represents k-th channel of an input feature map, * represents a convolution operation, and σ represents a ReLU activation function;
$${W}_{base}^{( k )}$$
represents a base weight of the k-th convolution kernel; α represents an adjustment coefficient configured to control an extent of influence of the ray model on a convolution kernel weight;
$${R}_{conv}^{( k )}$$
represents an adjusted weight guided by the ray model for the k-th convolution kernel; wᵢ represents a weight of i-th ray, Pᵢ represents a set of all points on the i-th ray,
$${R}_{i}^{( k )}$$
represents a feature value of the i-th ray in the k-th convolution kernel, and N represents a number of rays; Rᵢ(p) represents a feature value of the i-th ray at a point p, θ represents a hyperparameter for controlling a weight distribution, and Z represents a normalization factor.

3. The method of claim 1, wherein obtaining the fracture imaging data for the fracture region of the patient comprises:
receiving an original CT image, an original X-ray image, and an original light-field image respectively from a CT scanner, an X-ray device and a light-field camera;
performing contrast enhancement respectively on the original CT image, the original X-ray image, and the original light-field image to obtain an enhanced CT image, an enhanced X-ray image, and an enhanced light-field image;
processing the original CT image by using a contrast-limited adaptive histogram equalization:
$${I}_{CLAHE} ( x , y ) = \frac{L - 1}{MN} {\sum }_{k = 0}^{{I}_{1} ( x , y )} {h}_{clip} ( k )$$
wherein I₁(x,y) represents a pixel value of the original CT image at a position (x,y), I_{CLAHE}(x,y) represents a pixel value of the enhanced CT image at the corresponding position; L is a number of grayscale levels; M and N are width and height of an image respectively, h_{clip}(k) is a cumulative distribution function of a clipped histogram;
processing the original X-ray image by using adaptive contrast enhancement:
$${I}_{adaptive} ( x , y ) = {I}_{2} ( x , y ) \cdot ( 1 + \frac{{I}_{2} ( x , y ) - {μ}_{local} ( x , y )}{{σ}_{local} ( x , y ) + ò} )$$
wherein I₂(x,y) represents a pixel value of the original X-ray image at the position (x,y), I_{adaptive}(x,y) is a pixel value of the enhanced X-ray image at a corresponding position; μ_{local}(x,y) is a mean value of images at a local neighborhood of the position (x,y), σ_{local}(x,y) is a standard deviation of images in the local neighborhood of the position (x, y), and Ò represents a preset constant;
processing the original light-field image by using a multi-scale Retinex algorithm:
$${I}_{Retinex} ( x , y ) = {\sum }_{s} w ( s ) ( log {I}_{3} ( x , y ) - log ( {G}_{s} * I ) ( x , y ) )$$
wherein I₃(x,y) represents a pixel value of the original light-field image at the position (x,y), I_{adaptive}(x,y) is a pixel value of the enhanced light-field image at the corresponding position; w(s) represents a weight corresponding to scale s, and Gₛ denotes a Gaussian filter with the scale s;
detecting feature points in the CT image, the X-ray image, and the light-field image respectively based on a feature point detection algorithm, and performing feature point matching to register the enhanced CT image, the enhanced X-ray image, and the enhanced light-field image; and
determining the fracture imaging data of the fracture region of the patient based on the registered enhanced CT image, the registered enhanced X-ray image, and the registered enhanced light-field image.

4. The method of claim 3, wherein reconstructing the three-dimensional model of the fracture part based on the multimodal fusion feature comprises:
inputting the multimodal fusion feature into a three-dimensional convolutional neural network to generate an initial three-dimensional model of the fracture part;
inputting the initial three-dimensional model into a generative adversarial network to update the initial three-dimensional model and obtain the three-dimensional model of the fracture part, the generative adversarial network comprising a generator and a discriminator;
determining an importance of each position and adjust a weight of a feature map by introducing a channel attention module and a spatial attention module into a convolutional layer of the generator:
$$\begin{matrix}{F}_{CA} = Sigmoid ( FC 2 ( ReLU ( FC 1 ( GAP ( F ) ) ) ) ) \\ {F}_{SA} = Sigmoid ( Conv ( Concat [ AvgPool ( F ) ; MaxPool ( F ) ] ) )\end{matrix}$$
wherein F_{CA} represents a channel attention map, FC1 and FC2 are fully connected layers, and GAP is global average pooling; F_{SA} represents a spatial attention map; AvgPool and MaxPool are an average pooling operation and a max pooling operation respectively, and Concat denotes a feature concatenation operation;
a loss function L_{G} of the generator being defined as:
$$\begin{matrix}{L}_{G} = {L}_{gen} + {λ}_{pixel} {L}_{pixel} \\ {L}_{gen} = - log ( {D}_{attn} ( {G}_{attn} ( {V}_{initial} ) ) ) \\ {L}_{pixel} = {‖ {V}_{real} - {G}_{attn} ( {V}_{initial} ) ‖}_{1}\end{matrix}$$
wherein L_{gen} is a generator loss, Dₐₜₜₙ denotes a discriminator network, Gₐₜₜₙ denotes a generator network, and Vᵢₙᵢₜᵢₐₗ represents the initial 3D model of the fracture part; Lₚᵢₓₑₗ is a pixel-level reconstruction loss; λ·λ₁ represents a L1 norm; Vᵣₑₐₗ is a real 3D model of the fracture part; and λₚᵢₓₑₗ is a weight coefficient of the pixel-level reconstruction loss;
a loss function L_{D} of the discriminator being defined as:
$${L}_{D} = - log ( {D}_{attn} ( {V}_{real} ) ) - log ( 1 - {D}_{attn} ( {G}_{attn} ( {V}_{initial} ) ) ) .$$

5. The method of claim 4, wherein the AR glass is provided with an optical tracking module,
wherein aligning and calibrating the reconstructed three-dimensional model of the fracture part with the fracture region of the patient in the actual surgical scene comprises:
collecting marker coordinates of a plurality of markers based on the optical tracking module, each of the plurality of markers being preset in the fracture region of the patient according to a predefined marker position relationship;
determining a rotation matrix R and a translation vector t using a least square method based on the predefined marker position relationship:
$${min}_{R , t} {\sum }_{g} {‖ R \cdot {H}_{g} + t - {Q}_{g} ‖}^{2}$$
solving for an optimal R and t through a singular value decomposition, wherein H_{g} and Q_{g} respectively represent coordinates of g-th marker in the three-dimensional model of the fracture part and the actual surgical scene;
preliminarily aligning the three-dimensional model of the fracture part with the actual surgical scene by using rigid transformation based on the optimal R and t:
$${T}_{rigid} ( x ) = R \cdot x + t$$
wherein x represents an initial coordinate of any voxel point in the three-dimensional model of the fracture part, and T_{rigid}(x) represents a coordinate after rigid transformation;
determining a weight wᵤ of a control point pᵤ using a Laplacian matrix, and performing non-rigid transformation based on the weight wᵤ to adapt to a deformation and displacement of the fracture region of the patient:
$${T}_{non - rigid} ( x ) = x + {\sum }_{u = 1}^{N} {w}_{u} ϕ ( ‖ x - {p}_{u} ‖ )$$
wherein T_{non-rigid}(x) represents a coordinate after non-rigid transformation, and ϕ(r) denotes a Gaussian function;
fusing the coordinate T_{rigid}(x) after the rigid transformation and the coordinate T_{non-rigid}(x) after the non-rigid transformation using an adaptive adjustment algorithm to dynamically adjust an alignment state of the three-dimensional model:
$${T}_{adaptive} ( x ) = {T}_{rigid} ( x ) + β \cdot ( {T}_{non - rigid} ( x ) - {T}_{rigid} ( x ) )$$
wherein T_{adaptive}(x) represents a coordinate after the adaptive adjustment, and β denotes an adaptive adjustment coefficient.

6. An electronic device, comprising:
a processor;
a memory storing computer-readable instructions that, when executed by the processor, cause the electronic device to perform the method according to claim 1.

7. An electronic device, comprising:
a processor;
a memory storing computer-readable instructions that, when executed by the processor, cause the electronic device to perform the method according to claim 2.

8. An electronic device, comprising:
a processor;
a memory storing computer-readable instructions that, when executed by the processor, cause the electronic device to perform the method according to claim 3.

9. An electronic device, comprising:
a processor;
a memory storing computer-readable instructions that, when executed by the processor, cause the electronic device to perform the method according to claim 4.

10. An electronic device, comprising:
a processor;
a memory storing computer-readable instructions that, when executed by the processor, cause the electronic device to perform the method according to claim 5.

11. A non-transitory storage medium having a program code stored thereon that, when executed by a processor, the method according to claim 1 is performed.

12. A non-transitory storage medium having a program code stored thereon that, when executed by a processor, the method according to claim 2 is performed.

13. A non-transitory storage medium having a program code stored thereon that, when executed by a processor, the method according to claim 3 is performed.

14. A non-transitory storage medium having a program code stored thereon that, when executed by a processor, the method according to claim 4 is performed.

15. A non-transitory storage medium having a program code stored thereon that, when executed by a processor, the method according to claim 5 is performed.
